(19) United States
(12) Patent Application Publication
(10) Pub. No.: US 2015/0166596 A1
(43) Pub. Date: Jun. 18, 2015

(54) Retro Diels Alder Reaction as a Cleavable Linker in DNA/RNA Applications

(71) Applicant: Agilent Technologies, Inc., Loveland, CO (US)

(72) Inventor: Kenneth W. Hill, Lyons, CO (US)

(21) Appl. No.: 14/402,216

(22) PCT Filed: Apr. 29, 2013
(86) PCT No.: PCT/US2013/038704

§ 371 Date: Nov. 19, 2014

Related U.S. Application Data
(60) Provisional application No. 61/649,753, filed on May 21, 2012.

(51) Int. Cl. C07H 21/04

(52) U.S. Cl. CPC C07H 21/04

(57) ABSTRACT

The invention provides a novel approach for reversibly conjugating an oligonucleotide, which includes obtaining an oligonucleotide labeled with a diene moiety and a target entity labeled with a dienophile moiety; heating the oligonucleotide labeled with the diene moiety and the target entity labeled with the dienophile moiety in a solution at a first temperature to effect Diels Alder reaction to produce a conjugate; and heating the conjugate to a second temperature to effect retro Diels Alder reaction to regenerate the oligonucleotide labeled with the diene moiety and the target entity labeled with the dienophile moiety.

### PRIORITY CLAIMS AND RELATED PATENT APPLICATIONS

This application claims the benefit of priority from U.S. Provisional Application Ser. No. 61/649,753, filed on May 21, 2012, the entire content of which is incorporated herein by reference in its entirety.

### FIELD OF THE INVENTION

The present invention generally relates to the field of conjugating oligonucleotides to other entities. More particularly, the invention relates to compositions and methods wherein retro Diels Alder reaction is utilized as a cleavable linker in oligonucleotide applications.

### BACKGROUND OF THE INVENTION

Considerable efforts have been directed to the application of oligonucleotides and oligonucleotide analogs as diagnostic and research reagents and as potential therapeutics. As a result, the use of oligonucleotides as therapeutic agents or diagnostic agents is growing rapidly. In many of these applications, the oligonucleotides are conjugated with another molecular entity.

Most methods for attaching oligonucleotides (e.g., DNA or RNA) to other entities, such as small molecules, peptides, proteins, other oligonucleotides, polymers, and even solid surfaces, involve a tailored linker moiety that irreversibly attaches the oligonucleotides with the desired substance. For various chemical methods available to accomplish such conjugations, see a review by Goodchild, Bioconjugate Chemistry, 1:165-187 (1990). While these methods work well, most do not allow for subsequent removal of the oligonucleotides from the conjugation partner.

For example, many oligonucleotide based drugs have a large molecular weight PEG (40K) conjugated to them. Attachments of PEG may reduce the immunogenicity or increase the stabilities or half lives of the conjugates. Various methods for making PEG-oligonucleotide conjugates are described in Goodchild, Bioconjugate Chem., 1:165 (1990), and Zalipsky, Bioconjugate Chem., 6:150 (1995). The typical N-hydroxysuccinimide (NHS)-based conjugation methods used to make PEG-oligonucleotide conjugates produce non-cleavable linkers. The large sizes of the PEG-oligonucleotide conjugates prohibit mass spectral analysis, rendering it impossible to perform impurity characterization. In situations like this, it would be desirable to be able to reversibly attach and detach the oligonucleotides to the target entities.

In order to allow subsequent de-conjugation, other conjugation methods using cleavable linkers should be used. However, only thiol-disulfide based cleavable, reversible linkers are currently available. The thiol-disulfide based methods are widely used in protein conjugation chemistry as well as in oligonucleotide conjugation. Thiol-disulfide based conjugations do have some disadvantages. These linkers are unstable to many nucleophilic and electrophilic reagents. The conjugation requires a reducing agent to convert the oligonucleotide-S-SR to the reactive oligonucleotide-SH (i.e., a thiol). The oligonucleotide-SH species is oxidatively unstable; care must be taken to run reactions and manipulations under inert atmosphere to avoid oligonucleotide-S-S-oligonucleotide dimerization or reformation of the starting oligonucleotide disulfide (oligo-S-SR). Isolation or storage of the thiol labeled oligonucleotide is not practical due to its' oxidative instability.

In addition, the thiol-disulfide chemistry conjugates two species with the same conjugating group, SH. This can lead to mixed dimer formation during conjugation. Thus, there is a need for better methods that can reversibly conjugate oligonucleotides to other entities.

### SUMMARY OF THE INVENTION

One aspect of the invention relates to methods for reversibly conjugating an oligonucleotide. A method in accordance with one embodiment of the invention includes obtaining an oligonucleotide labeled with a diene moiety and a target entity labeled with a dienophile moiety; heating the oligonucleotide labeled with the diene moiety and the target entity labeled with the dienophile moiety in a solution at a first temperature to effect Diels Alder reaction to produce a conjugate; and heating the conjugate to a second temperature to effect retro Diels Alder reaction to regenerate the oligonucleotide labeled with the diene moiety and the target entity labeled with the dienophile moiety in ratios dependent upon temperature, diene and dienophile concentrations.

Another method in accordance with one embodiment of the invention includes obtaining an oligonucleotide labeled with a dienophile moiety and a target entity labeled with a diene moiety; heating the oligonucleotide labeled with the dienophile moiety and the target entity labeled with the diene moiety in a solution at a first temperature to effect Diels Alder reaction to produce a conjugate; and heating the conjugate to a second temperature to effect retro Diels Alder reaction to regenerate the oligonucleotide labeled with the dienophile moiety and the target entity labeled with the diene moiety in ratios dependent upon temperature, diene and dienophile concentration and whether excess of one was added to the retro Diels Alder reaction mixture.

Any method described above may be conducted in an aqueous solution, wherein the first temperature may be 20° C. and the second temperature may be 75° C. In any of the above methods, the diene moiety may comprise a furan and the dienophile entity may comprise a maleimide. In any of the above methods, the target entity may comprise a support, wherein the support may a solid support or a soluble polymer. In any of the above methods, the target entity may comprise a ligand for coupling to a support, wherein the ligand may comprise biotin and the support may comprise avidin or streptavidin.

Other aspects and advantages of the invention will be apparent from the following description and the appended claims.

### BRIEF DESCRIPTION OF DRAWINGS

FIG. 1 shows schematic illustrating a Diels Alder reaction and a retro Diels Alder reaction.

FIG. 2 shows a method for preparing a furan-labeled oligonucleotide in accordance with one embodiment of the invention.

FIG. 3 shows a Diels Alder reaction for conjugating an oligonucleotide derivative in accordance with one embodiment of the invention.

FIG. 4 shows a retro Diels Alder reaction for de-conjugating an oligonucleotide derivative in accordance with one embodiment of the invention.

FIG. 5 shows a Diels Alder reaction for conjugating an oligonucleotide derivative to facilitate purification by immobilization of a support and a retro Diels Alder reaction for de-conjugating the adduct to regenerate the oligonucleotide derivative in accordance with one embodiment of the invention.

FIG. 6 shows that the retro-Diels Alder furan-oligonucleotide can be used again in another Diels Alder reaction

FIG. 7 shows a flow chart illustrating a method in accordance with one embodiment of the invention.

### DEFINITIONS

Unless defined otherwise, all technical and scientific terms used herein have the same meaning as commonly understood by one of ordinary skill in the art to which this invention belongs. Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Sambrook et al., Molecular Cloning: A Laboratory Manual, 2.sup.nd Edition (Cold Spring Harbor Laboratory, 1989); and the like. Still, certain terms are defined below for the sake of clarity and ease of reference.

The term “nucleoside”, as used herein, refers to a modified or naturally occurring deoxyribonucleoside or ribonucleoside or any chemical modifications thereof. Modifications of the nucleosides include, but are not limited to, 2′-, 3′- and 5′-position sugar modifications, 5- and 6-position pyrimidine modifications, 2-, 6- and 8-position purine modifications, modifications at exocyclic amines, substitution of 5-bromo-uracil, and the like. Nucleosides can be suitably protected and derivatized to enable oligonucleotide synthesis by methods known in the field, such as solid phase automated synthesis using nucleoside phosphoramidite monomers, H-phosphonate coupling or phosphate triester coupling.

The term “nucleotide”, as used herein, refers to a modified or naturally occurring deoxyribonucleotide or ribonucleotide. Nucleotide is a nucleoside as defined above having one or several phosphates or substituted phosphates attached at the 5′-, 2′- or 3′-positions. Nucleotides typically include purines and pyrimidines, which include thymidine, cytidine, guanosine, adenine and uridine.

The “oligonucleotide”, as used herein, refers to a polynucleotide formed from a plurality of linked nucleotide units as defined above. The nucleotide units each include a nucleoside unit linked together via a phosphate linking group. The term oligonucleotide also refers to a plurality of nucleotides that are linked together via linkages other than phosphate linkages such as phosphorothioate linkages. The oligonucleotide may be naturally occurring or non-naturally occurring. In a preferred embodiment the oligonucleotides of this invention have between 1-1,000 nucleotides. Oligonucleotides may be synthetic or may be made enzymatically, and, in some embodiments, are 10 to 50 nucleotides in length. Oligonucleotides may include ribonucleotide monomers (i.e., may be oligoribonucleotides) or deoxyribonucleotide monomers. Oligonucleotides may be 10 to 20, 21 to 30, 31 to 40, 41 to 50, 51-60, 61 to 70, 71 to 80, 80 to 100, 100 to 150, 150 to 200, 200 to 500, or greater than 500 nucleotides in length, for example.

The terms “diene” or “diene moiety”, as used herein, refer to a molecule bearing two conjugated double bonds. The diene may even be non-conjugated, if the geometry of the molecule is constrained so as to facilitate a cycloaddition reaction (see, Cookson, J. Chem. Soc., 5416 (1964)). The atoms forming these double bonds can be carbon or a heteroatom or any combination thereof.

The terms “dienophile” or “dienophile moiety”, as used herein, refer to a molecule bearing an alkene group, or a double bond between a carbon and a heteroatom, or a double bond between two heteroatoms. A dienophile can be any group, including but not limited to, a substituted or unsubstituted alkene, or a substituted or unsubstituted alkyne. Typically, the dienophile is a substituted alkene of the formula C═C—Z or Z′-C═C—Z, wherein Z and Z′ are electron withdrawing groups, such as CHO, COR, COOH, CO-aryl, CN, NO₂. Other examples of dienophiles include compounds having the formula, R₂—C═X, wherein X is a heteroatom, selected from the group consisting of oxygen, nitrogen, phosphorus and sulfur. For example, molecules bearing a primary amino group, such as amino acids or a lysine containing peptide, can be converted to efficient dienophiles by reaction with formaldehyde to yield their corresponding iminium salts, which can undergo Diels Alder cycloaddition with a diene group under mild conditions in aqueous solvents.

### DETAILED DESCRIPTION OF THE INVENTION

Embodiments of the invention relate to methods for reversibly conjugating oligonucleotides with other molecular entities. The oligonucleotides may include DNA, RNA, or a mixed DNA/RNA. Embodiments of the invention are based on the fact that Diels Alder reactions (e.g., between furans, anthracenes and other suitable dienes, with maleimides and other suitable dienophiles) are reversible, and in many cases, readily reversible under mild conditions (e.g., by application of heat, light and pH change for example). Ready reversibility allows one to temporarily conjugate or de-conjugate the oligonucleotides.

For example, the difficulty noted above in performing mass spectrometer analysis on a PEG-conjugated oligonucleotide may be overcome by de-conjugation of the PEG from the oligonucleotides prior to mass spectrometer analysis. As a specific example, the conjugation of a furan-oligonucleotide with a maleimide modified PEG will give a stable conjugate that can be cleaved with applied heat, and the liberated furan oligonucleotide can be easily analyzed and impurities characterized LCMS methods. Thus, the problem of analysis of large molecular weight conjugates can be solved using the simple Diels Alder-retro Diels Alder reaction combination.

The use of Diels Alder reaction to conjugate molecules is known. For example, U.S. Pat. No. 6,737,236, issued to Pieken et al., discloses methods for conjugating macromolecules to other molecular entities using cycloaddition reactions, such as Diels Alder reaction or 1,3-dipolar cycloadditions. For Diels Alder reactions, a molecule containing a diene moiety (e.g., a furan) is coupled with another molecule containing a dienophile (e.g., a double bond containing moiety).

However, the use of retro Diels Alder reactions to release oligonucleotide conjugates of Diels Alder reactions has not been shown. Embodiments of the invention provide very efficient methods for temporarily conjugating oligonucleotides to target entities and for efficiently regenerating the oligonucleotide derivatives or creating new derivatives depending on the dienophile added in the second forward Diels Alder reaction.

Unlike sulfur-based cleavable and reversible linking chemistry, reagents for Diels Alder reaction (e.g., furans-labeled oligonucleotides) are stable and can be readily isolated. The Diels Alder reactions and the retro Diels Alder reactions described herein can use any suitable dienes and dienophiles. For example, embodiments of the invention may use furan or furan analogs as dienes and maleimide or its analogs as dienophiles. In Diels Alder reactions, the two groups that participate in the coupling are different (e.g., furan/maleimide (diene/dienophile)). Thus, one can avoid the unwanted dimer formation (as in the thiol-disulfide approach).

Embodiments of the invention involve Diels Alder reactions in aqueous media. It is known that Diels Alder reactions are more efficient in aqueous solutions (Rideout and Breslow, J. Am. Chem. Soc., 102:7816 (1980)). For example, simple dienes, such as sodium 3,5-hexadienoate and sodium 4,6-heptadienoate readily can undergo Diels-Alder reactions in water with a variety of dienophiles at ambient temperature. (Grieco et al., J. Org. Chem., 48:3137 (1983)), and the otherwise difficult cycloaddition of dimethyl acetylenedicarboxylate to an electron deficient furan proceeds under very mild conditions in water with very good yields. (Saksena et al., Heterocycles, 35:129 (1993)).

In some embodiments of the invention, the oligonucleotides may contain the diene moieties, while the target entities may comprise the dienophiles. In other embodiments, these may be reversed—i.e., the oligonucleotides may contain the dienophiles, whereas the target entities may contain the dienes. For clarity of illustration, the following description will use examples in which the oligonucleotides contain the dienes (e.g., furans), while the target entities contain the dienophiles (e.g., maleimides). One skilled in the art would appreciate that embodiments of the invention also include the reverse configuration (i.e., dienophiles on the oligonucleotides and the dienes on the target entities). Furthermore, the following examples may use furan as a diene and N-ethyl maleimide (NEM) as a dienophile. Again, the use of these specific molecules in the examples is for clarity of illustration and is not intended to limit the scope of the invention.

The Diels-Alder reaction is a [4+2] cycloaddition reaction; it involves a system of 4-π electrons (the diene) and a system of 2-π electrons (the dienophile). The reaction can occur rapidly under mild conditions, and the reaction can occur with a wide range of reactants. A review of Diels-Alder reaction can be found in: “Advanced Organic Chemistry,” (March. J., ed.) 761-798, McGraw Hill, N.Y. (1977). Diels Alder reactions are readily reversible, and the reverse reactions are referred to as retro Diels Alder reactions.

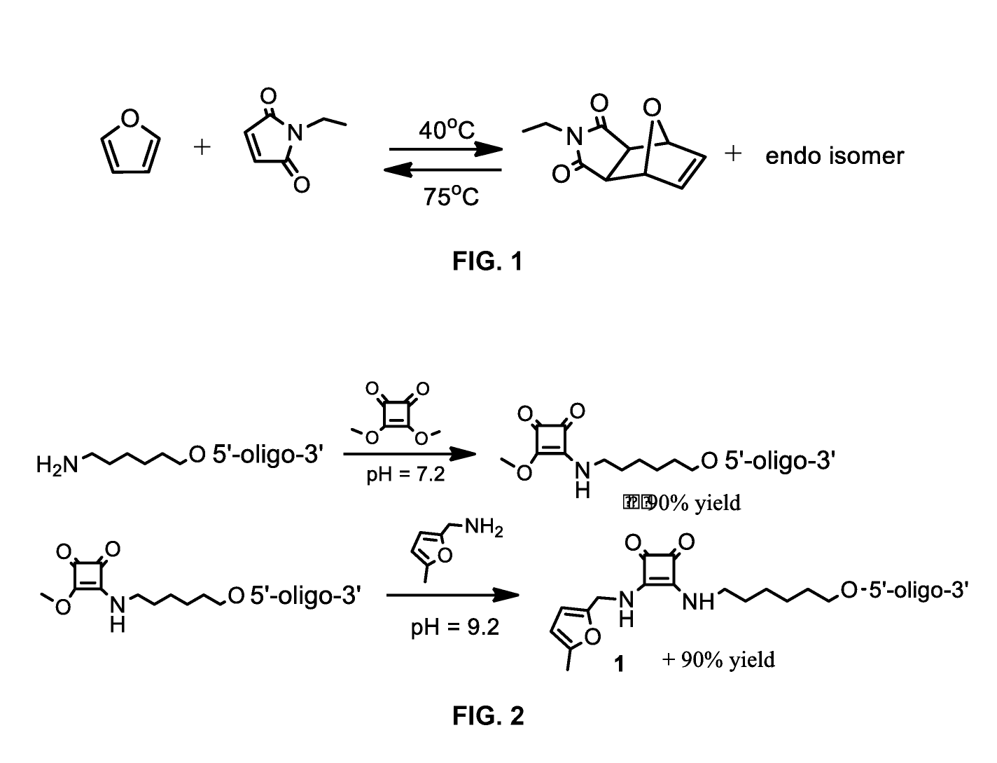

FIG. 1 shows an example of a basic Diels Adler reaction and the corresponding Retro Diels Alder reaction. In this example, furan acts as a diene component and N-ethyl maleimide (NEM) acts as a dienophile. The Diels Alder reaction may be catalyzed by heat. For example, the (4+2) cycloaddition reaction may be accomplished by heating the solution to 40° C. for a selected duration. The duration would depend on the specific dienes and dienophiles and the reaction medium used. One skilled in the art would be able to find the proper durations without undue experimentation. For example, one can use a suitable means to monitor the progress of the reaction and then termination the reaction when the reaction is complete or reaches the desired extent. The Diels Alder reactions may produce two stereoisomers (exo and endo isomers), either or both of which may be used with embodiments of the invention.

As noted above, the Diels Alder reactions are reversible. The retro Diels Alder reaction may also be catalyzed by heat. For example, the Diels Alder adducts may be dissociated by heating the solution to 75° C. for a selected duration. Again, one skilled in the art can easily determine the suitable duration without undue experimentations.

Embodiments of the invention make use of Diels Alder reactions, similar to that shown in FIG. 1, for conjugating oligonucleotides to target entities. The target entities may be any desired targets, such as other oligonucleotides, proteins/peptides, carbohydrates, or supports (which may include solid supports, such as resins, glass beads, magnetic beads, matrix surfaces, etc.). In accordance with some embodiments of the invention, oligonucleotides may be coupled to dienes, such as furans, while the target entities may be coupled to dienophiles, or vice versa. The diene-containing oligonucleotide derivatives can react with suitable dienophiles that are attached to the target entities under conditions similar to those shown in FIG. 1, such as by heating to 40° C. in an aqueous solution for a selected duration.

In accordance with embodiments of the invention, oligonucleotide derivatives (containing dienes or dienophiles) may be prepared with any suitable methods. For example, the oligonucleotides may be synthesized with a functional group for coupling with a diene or a dienophile. The functional groups, for example, may be amino groups, carboxyl groups, thiol groups, or the like. Alternatively, one may use the exocyclic amino groups on the nucleobases for the coupling.

Various methods for attaching functional groups to oligonucleotides are known. (For a review, see Goodchild, Bioconjugate Chemistry, 1:165-187 (1990)). Once the chemically reactive functional groups are attached to oligonucleotides (e.g., at the 5′-terminus), these reactive functional groups can be used to couple with various conjugates. For example, a primary aliphatic amino group may be incorporated at the 5′-terminus of the oligonucleotide in the final step of the synthesis of an oligonucleotide. Reagents for linking to the 5′ terminus of an oligonucleotide are commercially available. For example, 5′-Amino-Modifier C6 is available from Glen Research Corp. (Sterling, Va.).

The reagents used to modify the oligonucleotides to provide reactive functional groups may be in the form of phosphoramidites, which may be coupled to the free 5′-hydroxyl group of the full length oligonucleotide while it is attached to a solid support. This coupling would be like attaching another nucleotide monomer. For an example, please see Theison et al., Tetrahedron Lett., 33:5033-5036 (1992).

Once the oligonucleotides are derivatized with reactive functional groups (e.g., amino or thiol groups), they may be used to couple with a diene or a dienophile, either directly or via another intermediary.

FIG. 2 shows an example for preparing a furan-labeled oligonucleotide via an intermediary (e.g., squarate (SQ)). While an SQ is shown in this example, any suitable intermediaries known in the art may be used. As shown in FIG. 2, SQ may be coupled to an amino group under very mild conditions, such as in water at neutral pH. Mild reaction conditions are desirable for coupling biological materials, including oligonucleotide. In this coupling, excess SQ may be used to favor mono-substitution on the SQ moiety (i.e., only one of the two potential amino-reactive sites on SQ is coupled to an oligonucleotide).

After SQ is coupled to the amino-linked oligonucleotide, the excess SQ can be removed by any means know in the art. For example, because SQ is a small molecule, it can be separated based on molecular sizes, such as size exclusion gel filtration or molecular weight cutoff membrane filtration.

In this example, after coupling, the reaction mixture may be worked up to remove the small molecule species (excess squarate and small oligonucleotide failure species) with ultra filtration, for example using 3K molecular weight cutoff membranes in Epindorf tubes (or larger ultra filtration units for larger scale conjugations). With the ultra filtration, small molecule components/impurities may be quickly removed by centrifugation.

To produce a furan-coupled derivative, the mono-conjugate of SQ-oligonucleotide may be treated with excess 5-methyl furfuryl amine. Again, this coupling reaction can be conducted under mild conditions, such as in aqueous buffer at pH 9.2. The reaction may be again worked up using a molecular weight cutoff membrane (e.g., a 3K molecular weight cutoff membrane). The final retentate may be dried (e.g., lyophilized) to give a furan-SQ-oligonucleotide, 1. The final product 1 was confirmed by LCMS, and the yield was found to be greater than 90% as judged by liquid chromatography-mass spectrometry (LCMS) analysis.

The above example shows a furan is linked to an oligonucleotide via a squarate (SQ), which permits coupling using amino groups on both the oligonucleotides and the furans. One skilled in the art would also appreciate that an alternative is to have a furan derivative directly coupled with an amino-labeled oligonucleotide. For example, a furan derivative may contain a functional group for reaction with an amino group on the oligonucleotide derivative. Such functional groups may include activated carboxyl esters (e.g., an NHS ester), anhydride, acyl halides, aldehydes, or halides. These types of chemical modifications are well known in the art.

Furthermore, while the above example uses an amino-labeled oligonucleotide to react with an intermediary (e.g., a squarate) for coupling to a furan derivative, other oligonucleotides may contain other functional groups. For example, oligonucleotides of the invention may be derivatized with an amino-reactive group (e.g., a carboxyl group, an aldehyde group or a halide) that may be used to couple with a furan derivative that contains an amino group. One skilled in the art would appreciate that various modifications and variations are possible without departing from the scope of the invention.

Once furan-containing oligonucleotides are available, they may be used to conjugate with a target molecule that contains a dienophile moiety. Common dienophiles typically comprise a double bond, particularly a double bond adjacent to an electron withdrawing group. Dienophiles commonly used with biological molecules include maleimide derivatives, such as N-ethyl maleimide (NEM).

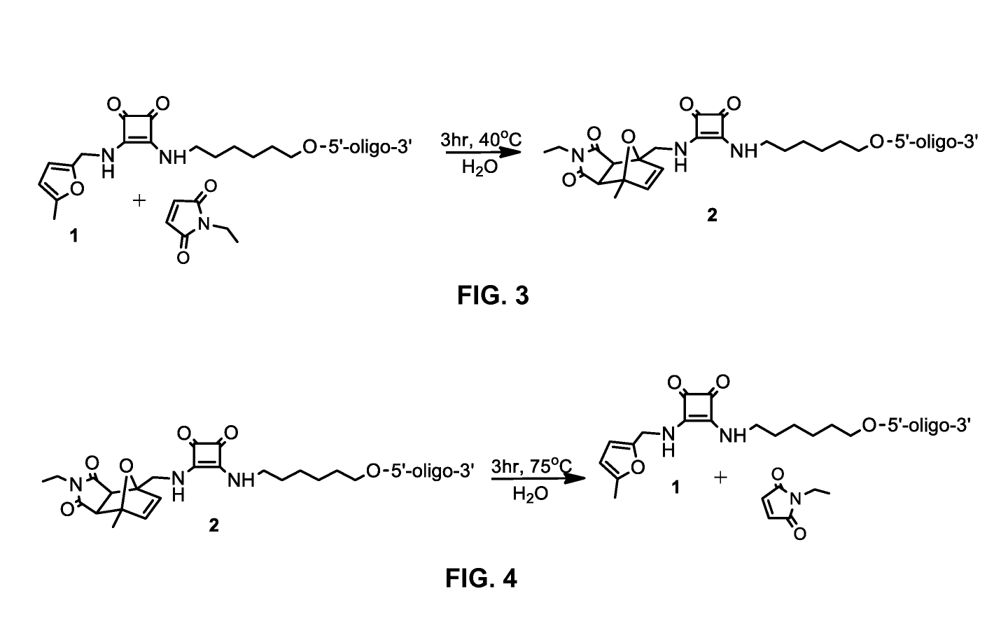

FIG. 3 shows an example of conjugating a furan-containing oligonucleotide to a dienophile (i.e., NEM). In this example, the isolated furan-SQ-oligonucleotide, 1, was dissolved in water, and an excess of N-ethyl maleimide (NEM) dissolved in dimethyl sulfoxide (DMSO) was added. The resultant mixture was kept at 40° C. After three hours at 40° C., the reaction mixture was sampled and analyzed directly by LCMS. The reaction had proceeded cleanly to give the expected Diels Alder adduct (probably a mixture of exo and endo isomers, but only the exo isomer is shown in FIG. 3) in greater than 90% yield. Again, the Diels Alder adduct 2 may be isolated by the ultra filtration (UF) spin cartridge method described above.

While the above example uses an ethyl maleimide (NEM) as a dienophile, other dienophiles may also be used. For example, the NEM derivatives may be linked to the target molecules that are to be coupled with oligonucleotides. In this case, when the Diels Alder reaction occurs between a furan-oligonucleotide and a dienophile-target molecule, the oligonucleotide and the target molecule will be covalently linked.

The dienophile and the target molecule may be directly linked or via a linker. Any suitable linkers may be used for this purpose, such as an alkyl based linker, a PEG linker, etc. One skilled in the art would appreciate that embodiments of the invention are not limited to any specific linkers for this purpose.

Once an oligonucleotide is covalently linked with a target molecule, they may be used for the intended purposes (e.g., to facilitate detection or purification). Once they serve their purposes, the conjugates may be reversed, via a retro Diels Alder reaction, to release the oligonucleotides from the target molecules.

An example of a retro Diels Alder reaction is illustrated in FIG. 4. As shown, a solution of the Diels Alder adduct (e.g., the retentate of the ultrafiltration (UF) purification after the synthesis shown in FIG. 3), which may be diluted to a proper volume (e.g., 500 μL). The solution was heated to 75° C. to effect the retro Diels Alder reaction. The reaction was sampled over time and analyzed by LCMS. After 3 hours at 75° C., the retro Diels Alder reaction was complete, and the furan-oligonucleotide 1 was produced in greater than 90% yield.

If recovery of the oligonucleotide derivative is desirable, the reaction mixture may be worked up with any suitable means, such as ultrafiltration using a 3K UF spin cartridge described above. The retentate from the filtration may be lyophilized to obtain the oligonucleotide derivative. The lyophilized solid was taken up in water and analyzed by LCMS. The LCMS data from this analysis revealed that the furan-SQ-oligonucleotide is recovered.

In order to show the truly reversible nature of the reaction between the furan derivatives and the dienophile derivatives (NEM), an aqueous solution of 1 (recovered from the above retro Diels Alder reaction) was again treated with excess NEM in DMSO. This mixture was heated at 40° C. After three hours, the mixture was analyzed by LCMS. As hoped for, the retro Diels Alder product 1 was smoothly converted back to the Diels Alder product, 2 (Reaction Scheme 3).

These examples show that the furan-SQ-oligonucleotide 1 not only undergoes the Diels Adler cycloaddition reaction in good yield (a requirement in linker performance), but the Diels Alder product 2 is also able to be converted back to 1 by just heating to 75° C. for a short period of time (a requirement of a good cleavable linker). Once the starting furan has been released from the Diels Alder adduct 2 (essentially a protected furan and maleimide), it can be used again in a conjugation reaction. These examples clearly show that these furan-oligonucleotide derivatives can be used to reversibly link or label a target molecule.

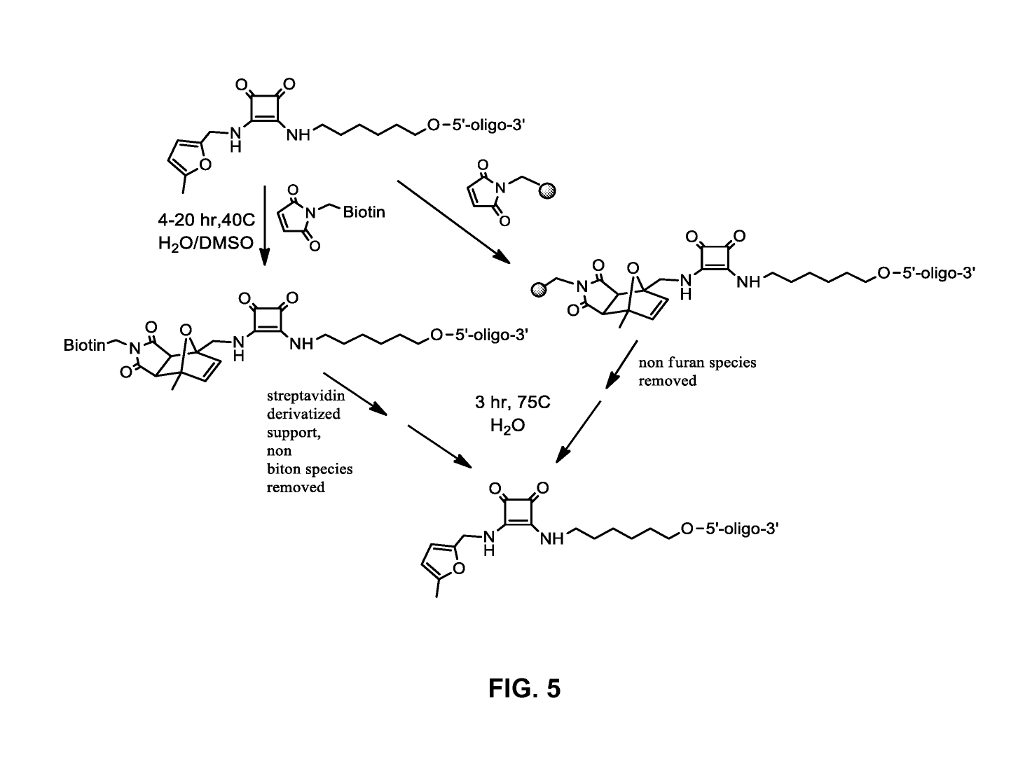

Being able to conjugate and de-conjugate oligonucleotides efficiently enables embodiments of the invention to be used in many applications. For example, these methods may be used to purify oligonucleotide derivatives. FIG. 5 shows an example of such an application.

Embodiments of the invention may be used to covalently link an oligonucleotide to a target. As illustrated in FIG. 5, a target entity may be a support for immobilizing the oligonucleotides. In one approach the NEM derivative may be linked directly to a support (e.g., soluble polymer, solid polymer, a magnetic bead, a solid surface, or resin). After Diels Alder reaction, the oligonucleotides will be bound to the solid support and the unbound impurities may be washed away. Then, the desired oligonucleotide derivative may be recovered by heating the solid support (e.g., at 75° C.) to effect the retro Diels Alder reaction.

In an alternative approach, the NEM derivative may be bonded to a ligand for coupling to a support (e.g., biotin as a ligand for coupling to a support having streptavidin or avidin). After Diels Alder reaction, the adduct (or conjugate) may be treated with a streptavidin derivatized support (e.g., soluble polymer, solid polymer, a magnetic bead, a solid surface, or resin. This will immobilize the oligonucleotide derivative on the support. After washing away the unbound impurities, the desired oligonucleotide may be released retro Diels Alder reaction (e.g., by heating at 75° C.).

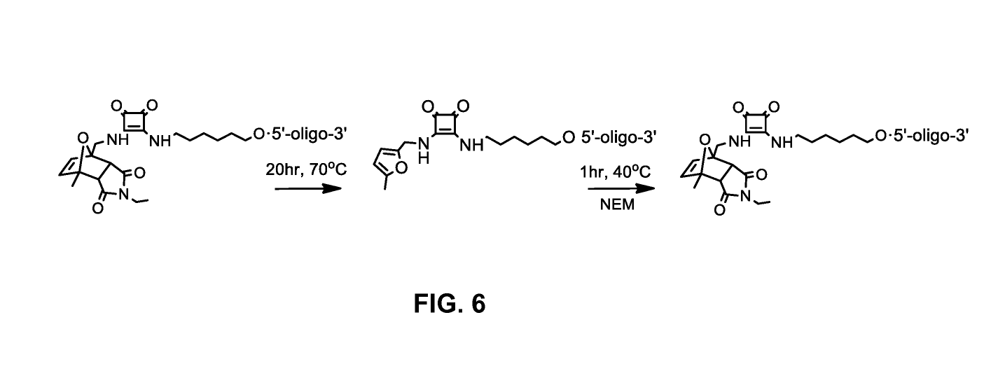

FIG. 6 Shows that the liberated furan labeled oligonucleotide by the retro Diels Alder reaction can be used in another Diels Alder reaction with the same or different or mixtures of dienophiles.

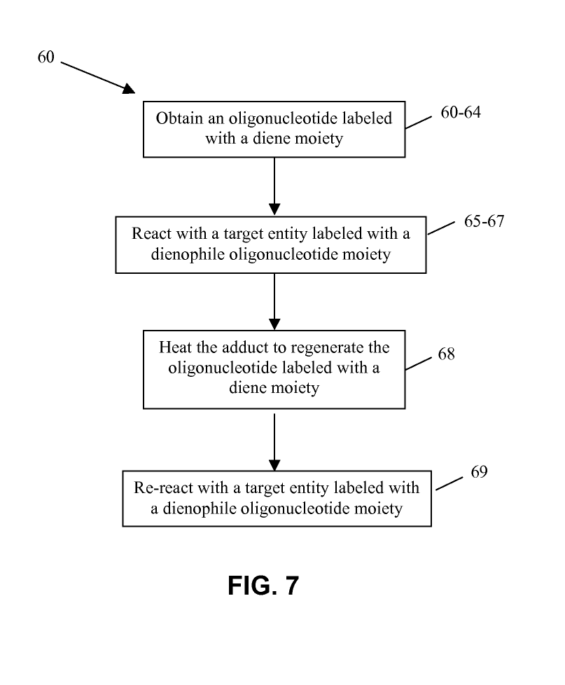

FIG. 7 shows a flow chart illustrating a method of the invention. As shown, a method 60 comprises obtaining an oligonucleotide labeled with a diene moiety (step 61). Then, react this oligonucleotide derivative with a target entity labeled with a dienophile moiety to effect Diels Alder reaction (step 62). Afterwards (e.g., after the adduct has served the intended purpose), the adduct is heated at a temperature to effect retro Diels Alder reaction to regenerate the oligonucleotide labeled with the diene moiety (step 63). The recovered diene-oligonucleotide can be converted back to a Diels Alder adduct by treating with the same or another dienophile. While the above example shows an oligonucleotide labeled with a diene moiety, it is also possible to use an oligonucleotide labeled with a dienophile to couple with a target entity labeled with a diene moiety.

### EXAMPLES

### Furan-Oligonucleotide Synthesis

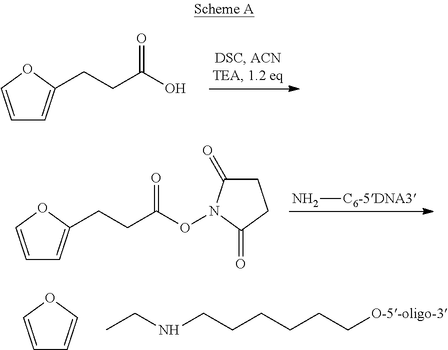

1.5 grams of the furan carboxylic acid was dissolved in 40 mL of ACN. To this was added 2.3 mL of triethyl amine followed by 3.0 grams of Disuccinimidyl carbonate (DSC). This mixture was stirred at room temperature for 3.5 hours. Analysis of the reaction mixture by TLC showed a new spot (KMnO₄ stain) and little to no remaining furan carboxylic acid. The ACN was removed by rotary evaporation and remaining oil taken up in methylene chloride and washed twice with 0.5 M sodium bicarbonate and once with 0.5 M NaCl. The methylene chloride solution was dried over magnesium sulfate, filtered and evaporated. The resulting solid was purified by Silica gel chromatography (60/40 hexanes/ethyl acetate). The product containing fractions were combined and rotary evaporated to approximately 20 mL. Hexanes were added drop wise to this solution until a white solid began to crystalize. This mixture was placed at 4° C. overnight. The crystalized material was filtered to give 1.5 grams after drying under vacuum (72% yield). ¹HNMR analysis showed that the product was the desired furan NHS ester.

An oligonucleotide (RNA 20mer or DNA 27mer) with a 5′ amine prepared as described above in 25 mM sodium borate, pH=9.2, at approximately 10 μM, was treated with an excess of the furan NHS ester in ACN, Scheme A. This resulted in complete conversion of the starting 5′ amino labeled oligonucleotide species to the furan-oligonucleotide as shown by LCMS analysis. The resulting furan-oligonucleotide was ultrafiltered (NaCl exchanged and diafiltered against water) then lyophilized.

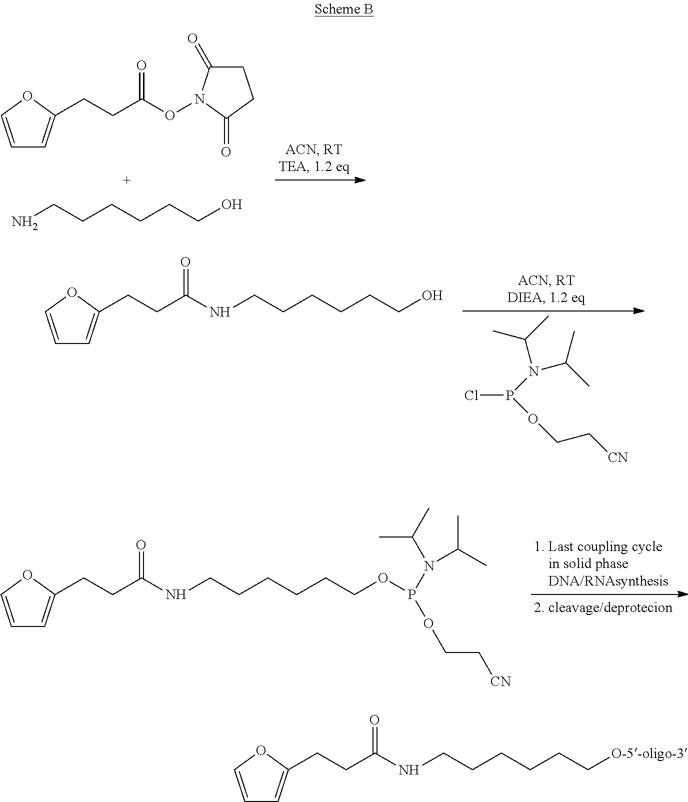

2.6 grams of the furan NHS ester was dissolved in 40 mL of ACN. To this was added 1.6 mL of triethyl amine followed by 1.5 grams of the amino hexanol. This mixture was stirred at room temperature for 2 hours. Analysis of the reaction mixture by TLC showed a new spot (KMnO₄ stain) and no remaining furan carboxylic acid NHS ester. The ACN was removed by rotary evaporation and remaining oil taken up in ethyl acetate and washed twice with 0.5 M sodium bicarbonate and once with 0.5 M NaCl. The ethyl acetate solution was dried over magnesium sulfate, filtered and evaporated to give 2.6 grams (96% yield) of and off white solid. ¹HNMR analysis showed that the product was the desired product.

The furan phosphoramidite was synthesized as shown in Scheme B. To 2.3 grams of the furan alcohol in a 100 mL round bottom flask was added 70 mL of ACN, this mixture was gently stirred and warmed until the furan alcohol was dissolved. To this stirring solution was slowly added 32/grams of diisopropylethyl amine followed by 3.2 grams of N, N′-diisopropyl phosphoramidous chloride. This mixture was stirred for 4 hours at room temperature. The ACN was removed by rotary evaporation and remaining oil taken up in methylene chloride and washed twice with 0.5 M sodium bicarbonate and once with 0.5 M NaCl. The methylene chloride solution was dried over magnesium sulfate, filtered and evaporated. The resulting oil was purified by Silica gel chromatography using ethyl acetate. The product containing fractions were combined and rotary evaporated to give 3.6 grams (85% yield) of a brown oil, single spot by TLC. ³¹PNMR showed only one peak at 147 ppm in agreement with the furan phosphoramidite. ¹HNMR analysis also confirmed that the oil was the desired furan phosphoramidite.

The furan amidite was used as the last amidite, 5′ end, in a solid phase oligonucleotide synthesis of an RNA 20mer, a DNA 27mer a DNA 57mer and an RNA 93mer. The furan labeled oligonucleotides (20, 27 and 57 mers) were cleaved and the DNA was deprotected using concentrated ammonia and or aqueous methyl amine, and the RNA 20mer was deprotected in concentrated ammonia followed by TEA 3HF. The 93 mer RNA made using TC protected RNA phosphoramidites, was deprotected in ethylene diamine. Analysis of the crude mixtures by LCMS showed that the furan amidite had coupled in excellent yield (>95%) and was stable to the deprotection conditions.

Diels Alder Conjunction of Maleimides with Furan-Oligonucleotides

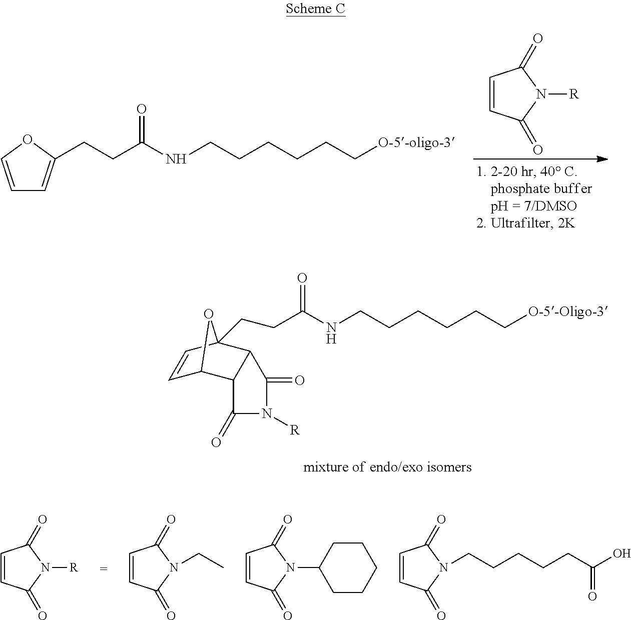

Diels Alder Reaction with N-ethyl maleimide

To an aqueous sodium phosphate buffered solution (pH=7) of furan-DNA 27mer, 2.3 mM, was added excess N-ethyl maleimide dissolved in DMSO (5% by total volume). This mixture was placed at 40° C. for 2 hours, see Scheme C. LCMS analysis of the reaction mixtures showed that the furan-oligonucleotide had been converted to the Diels Alder adducts in greater than 95% yield.

Diels Alder Reaction with N-cyclohexylmaleimide

To an aqueous sodium phosphate buffered solution (pH=7) of furan-DNA 27mer, 1.3 mM, was added excess N-ethyl maleimide dissolved in DMSO (70% by total volume). This mixture was placed at 40° C. for 20 hours, see Scheme C. LCMS analysis of the reaction mixtures showed that the furan-oligonucleotide had been converted to the Diels Alder adducts in approximately 90% yield.

Diels Alder Reaction with 6-maleimidohexanoic acid

To an aqueous sodium phosphate buffered solution (pH=7) of furan-DNA 27mer, 5 mM, was added excess N-ethyl maleimide dissolved in DMSO (10% by total volume). This mixture was placed at 40° C. for 5 hours, see Scheme C. LCMS analysis of the reaction mixtures showed that the furan-oligonucleotide had been converted to the Diels Alder adducts in greater than 95% yield.

Retro Diels Alder of Maleimide-Furan-Oligonucleotides Conjugates and Re Conjugation with Maleimides

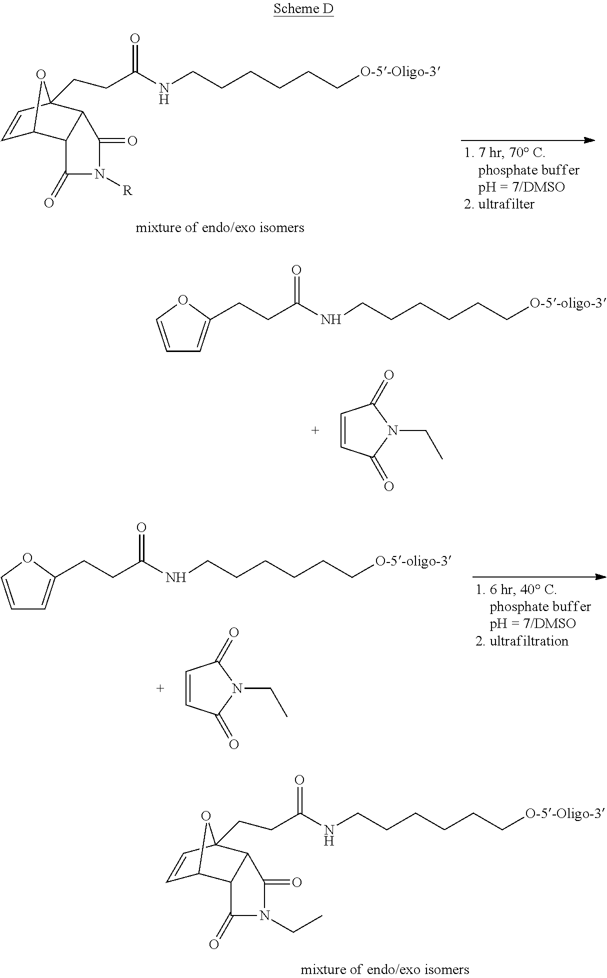

### Retro Diels Alder Reaction of Furan-DNA-Maleimide Diels Alder Conjugates.

The Diels Alder reaction mixture was ultrafiltered against waster using Amicon 3K spin filters. This removed the excess maleimide. The ultrafiltered solution of the furan-oligonucleotide N-ethyl maleimide Diels Alder product, at approximately 0.5 mM, was heated at 70° C., first reaction in Scheme D. LCMS analysis showed that after 7 hours of heating all the Diels Alder adducts had been converted back to the original furan-oligonucleotide. This mixture was ultrafiltered using Amicon 3K spin filters to remove the liberated N-ethyl maleimide.

Second Diels Alder Reaction with N-Ethyl Maleimide

To either the ultrafiltered retro Diels Alder reaction mixture or to a lyophilized sample of the ultrafiltered retro Diels Alder reaction mixture taken up in 100 mM sodium phosphate, pH=7, (5-1 mM furan oligonucleotide concentration), was added an excess of N-ethyl maleimide (NEM) in DMSO (5% DMSO by volume). After heating at 40° C. for 3-18 hours (depending on concentration) the regenerated furan-oligonucleotide had been completely converted back to the N-ethyl maleimide Diels Alder adducts as shown by LCMS analsysis, see Scheme D.

### In-Situ Generation of a Different Diels Alder Adduct by Conducting the Retro-Diels Alder Reaction in the Presence of Another Maleimide Species.

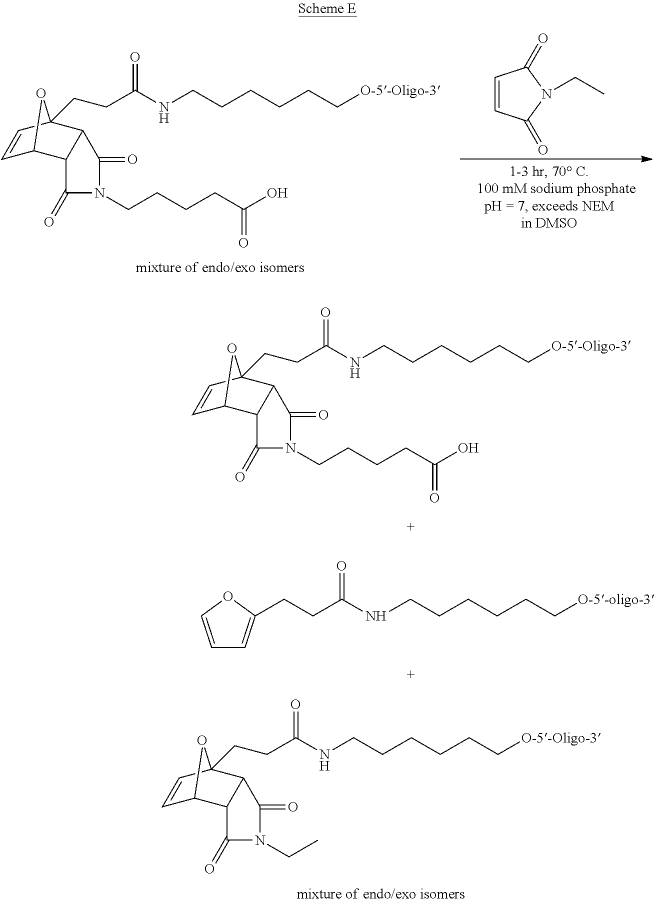

### Formation and Isolation of Initial Diels Alder Adducts Followed by Retro Diels Alder at 70° C. In the Presence of Excess N-Ethyl Maleimide

200 mg of furan DNA 27mer was dissolved in 2.5 mL of 100 mM sodium phosphate, pH=7. To this was added 50 mg of maleimide hexanoic acid dissolved in 100 μL of DMSO. This clear solution was placed at 40° C. LCMS analysis after 3 hours showed that the reaction had gone to completion (94%). The diluted reaction mixture was ultrafiltered against water using a 2K Hydrosart ultrafiltration membrane. The retentate was lyophilized to give 180 mg of a white solid. LCMS analysis gave the expected m/z and chromatographic profiles of the Diels Alder adducts.

6 mg of the lyophilized solid was dissolved in 150 μL of 100 mM sodium phosphate, pH=7. To this clear solution was added 20 uL of DMSO containing 5 mg of N-ethyl maleimide. This mixture was heated at 70° C. LCMS analysis after 1 hour showed that the reaction mixture consisted of approximately 10% furan-DNA 27mer, 50% of the starting furan-DNA 27mer-maleimide hexanoic acid Diels Alder adducts and 40% of the new N-ethyl maleimide Diels Alder adduct. After 3 hours at 70° C. the relative amounts of these species was approximately 10% furan-DNA 27mer, 40% of the starting furan-DNA 27mer-maleimide hexanoic acid Diels Alder adducts and 50% of the new N-ethyl maleimide Diels Alder adduct. See Scheme E.

### Oligonucleotide Purification by Diels Alder/Retro Diels Alder Reaction Using Biotin Maleimide and Streptavidin Agarose

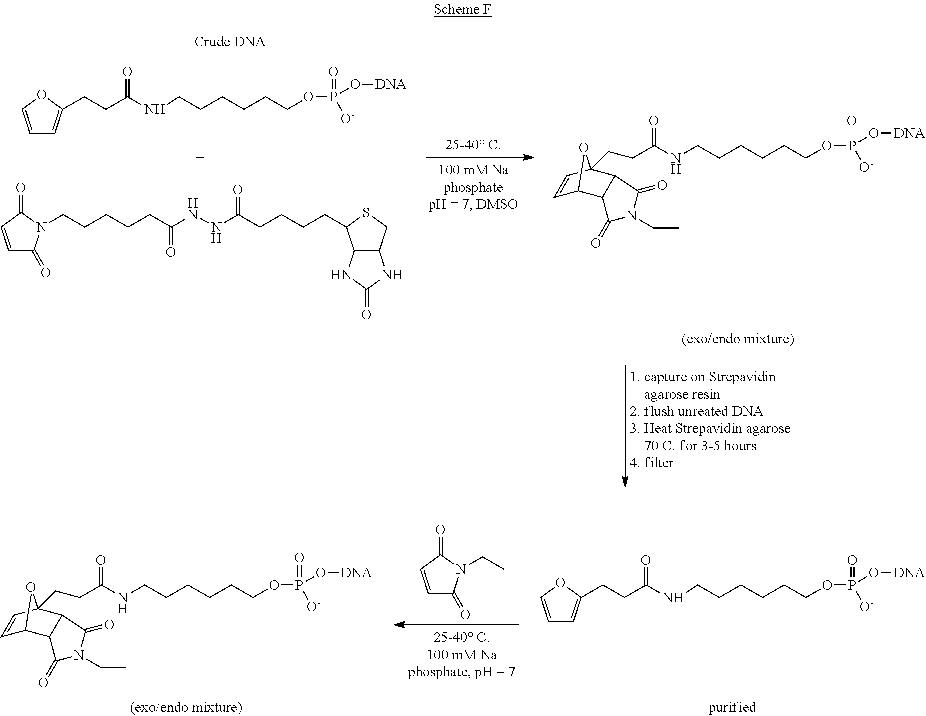

An ultrafiltered (salt exchange/water diafiltration) aqueous solution (phosphate buffer, pH=6-7) of crude furan-oligonucleotide, see Scheme F, was mixed with excess biotin maleimide dissolved in DMSO. This solution was placed at 40° C. overnight (15 hr.). LCMS analysis of the reaction solution showed that the Diels Alder reaction had gone to >90% conversion. The reaction mixture was ultrafiltered using a 3K membrane to remove unreacted biotin maleimide and all other small molecule species.

The resulting ultrafiltered oligonucleotide (Ultrafilter retentate) was added to excess streptavidin on agarose solid support. This mixture was let stand at approximately 25° C. for 4 hours. The solid support was filtered and washed with water until no UV active material eluted. LCMS analysis of the filtrate showed only synthesis failures and very small amounts of furan-oligonucleotide that did not undergo the Diels Alder reaction with the biotin maleimide.

The washed agarose solid support was taken up in water and heated to 70° C. for 8 hours. Filtration and washing of the support produced the purified furan-oligonucleotide with all non-furan containing species removed in approximately 80% recovery of furan-oligonucleotide.

The liberated furan-DNA obtained from the retro Diels Alder reaction, was made 1 m M, in 100 mM sodium phosphate, pH=7, was added excess NEM dissolved in DMSO (10% by volume). This mixture was heated at 40 C for 6 hours. LCMS analysis showed that all the furan-DNA had been re converted to the expected Diels Alder adducts.

Diels Alder and Retro Diels Alder Reactions of a PEG Maleimide with Furan-Oligonucleotides

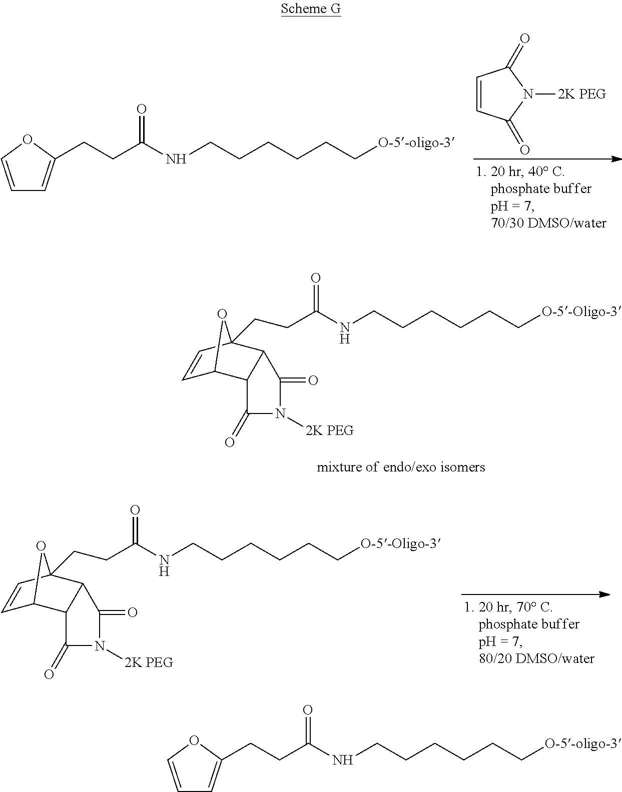

To 5 mg of lyophilized furan-DNA 27mer, see scheme G, was dissolved in 100 uL of in 100 mM sodium phosphate, pH=7. 20 mg of 2K PEG maleimide was dissolved in 200 uL of warm DMSO. The furan oligo solution was added to the PEG mixture and the resulting clear solution heated at 40° C. for 48 hours. This mixture was analyzed by anion exchange HPLC which showed that the 2 K PEG Diels Alder products had been formed.

The reaction mixture was diluted by 20 fold with water. This mixture was heated to 70° C. for 20 hours. Analysis of this solution by anion exchange HPLC showed that the 2K PEG Diels Alder adduct had reverted back to the furan-oligo 27mer.

### Oligonucleotide Purification by Diels Alder/Retro Diels Alder Reaction Using Maleimide Labeled Silica

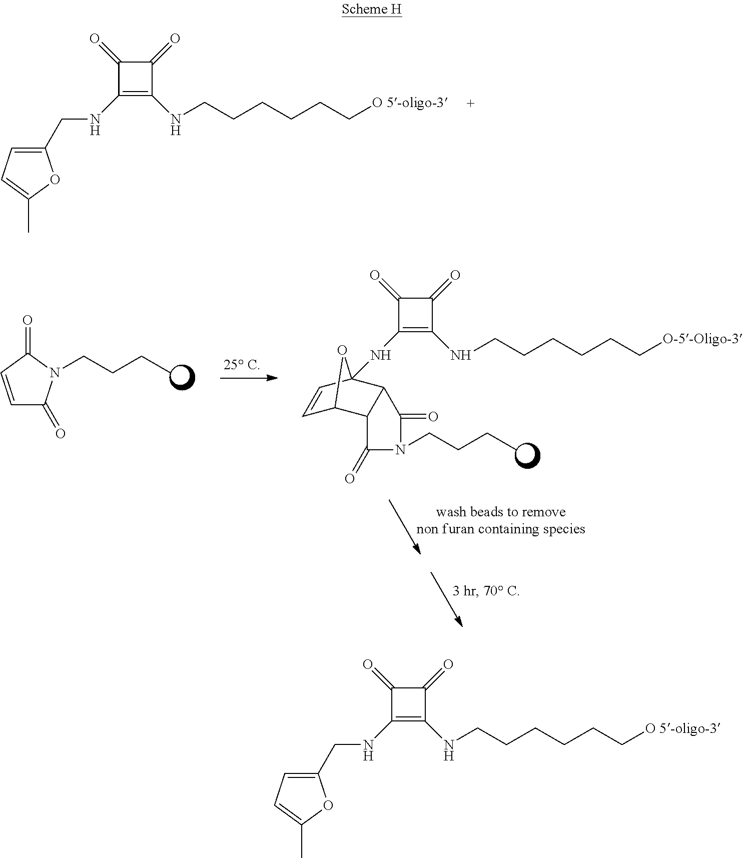

Dissolved 2.5 mg of the methyl furan oligonucleotide derivative, show in the above Scheme H, in 200 uL 200 mM sodium phosphate, pH=7.0. To this clear solution was added 70 mg of the maleimide-silica solid. This suspension was place at 25° C. for 18 hours. LCMS analysis of a sample of the reaction mixture showed that not much methyl furan labeled oligonucleotide remained; the mixture contained only non-furan labeled oligonucleotides.

The reaction mixture was diluted and the beads removed by filtration. The beads were washed with water and water methanol solutions until no UV active material was seen in the washes. The resin was then taken up in 300 uL of water+200 uL of methanol. This mixture was heated at 70° C. After three hours the reaction mixture was analyzed by LCMS, which showed the washings to contain only the methyl furan-oligonucleotide.

While the above examples are illustrated with a furan linked to an oligonucleotide, one skilled in the art would appreciate that other dienes may be used (instead of a furan) without departing from the scope of the invention. Furthermore, while the above examples are shown with a diene on an oligonucleotide and a dienophile linked to a target, embodiments of the invention also include oligonucleotides labeled with dienophiles while the targets are modified with dienes. While the above examples are shown with only one furan attached to the oligonucleotide, multiple attachments of dienophiles and or dienes (protected from reacting internally unless desired) to oligonucleotides can be performed, which can allow so labeled oligonucleotides to be used in applications where multiple forward and retro Diels Alder reactions are occurring.

Advantages of embodiments of the invention may include one or more of the following. Embodiments of the invention provide efficient methods for reversibly conjugate oligonucleotides using Diels Alder reactions and retro Diels Alder reactions. The reactions can be conducted under mild conditions and the yields are very good. For example, Diels Alder reactions may be effected by simply warming a solution of the two reactants to 20° C. and the retro Diels Alder reaction may be effected by heating the Diels Alder adduct to 75° C. The liberated furan oligonucleotide can be isolated and used again repetitively or with a different maleimide derivative. This ability to isolate and re-isolate the oligonucleotide derivatives provides a simple means of changing the conjugation group.

By controlling the concentrations of dienophiles in solution, the furan-oligonucleotide, of furan-oligonucleotide-maleimide Diels Alder product the ratio of starting furan-oligonucleotide along with the possible Diels Alder adducts (dependent on the number and concentration of dienophiles present). The dynamic of this reaction can be used in oligonucleotide based combinatorial chemistry applications as well as oligonucleotide based devices and assays.

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples disclosed herein are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance that can be adapted to the practice of this invention in its various embodiments and equivalents thereof.

What is claimed is:

1. A method for reversibly conjugating an oligonucleotide, comprising:
obtaining an oligonucleotide labeled with a diene moiety and a target entity labeled with a dienophile moiety;
heating the oligonucleotide labeled with the diene moiety and the target entity labeled with the dienophile moiety in a solution at a first temperature to effect Diels Alder reaction to produce a conjugate; and
heating the conjugate to a second temperature to effect retro Diels Alder reaction to regenerate the oligonucleotide labeled with the diene moiety and the target entity labeled with the dienophile moiety.

2. The method of claim 1, wherein the solution is an aqueous or aqueous/organic solution.

3. The method of claim 1, wherein the first temperature is about 20° C. and the second temperature is about 75° C.

4. The method of claim 1, wherein the diene moiety comprises a furan group.

5. The method claim 1, wherein the dienophile entity comprises a maleimide group.

6. The method of claim 1, wherein the target entity comprises a support.

7. The method of claim 6, wherein the support is a solid support, gel, nanoparticle, protein, carbohydrate, soluble polymer or mixture thereof.

8. The method of claim 1, wherein the target entity comprises a ligand for coupling to a support.

9. The method of claim 8, wherein the ligand comprises biotin and the support comprises avidin or streptavidin.

10. The method of claim 1, wherein the oligonucleotide and the diene moiety are linked via a squarate group.

11. A method for reversibly conjugating an oligonucleotide, comprising:
obtaining an oligonucleotide labeled with a dienophile moiety and a target entity labeled with a diene moiety;
heating the oligonucleotide labeled with the dienophile moiety and the target entity labeled with the diene moiety in a solution at a first temperature to effect Diels Alder reaction to produce a conjugate;
heating the conjugate to a second temperature to effect retro Diels Alder reaction to regenerate the oligonucleotide labeled with the dienophile moiety and the target entity labeled with the diene moiety; and
re-labeling the liberated furan-oligonucleotide with the same or other dienophile(s); and
optionally repeating the process of retro Diels Alder with Diels Alder conjugations-de-conjugation reactions.

12. The method of claim 11, wherein the solution is an aqueous or aqueous/organic solution.

13. The method of claim 11, wherein the first temperature is about 20-40° C. and the second temperature is about 75° C.

14. The method of claim 11, wherein the diene moiety comprises a furan group.

15. The method of claim 11, wherein the dienophile entity or entities, comprises a maleimide group.

16. The method of claim 11, wherein the target entity comprises a support.

17. The method of claim 16, wherein the support is a solid support, gel, nanoparticle, protein, carbohydrate soluble polymer or mixture thereof.

18. The method of claim 11, wherein the target entity comprises a ligand for coupling to a support.

19. The method of claim 18, wherein the ligand comprises biotin and the support comprises avidin or streptavidin.

20. The method of claim 11, wherein the oligonucleotide and the dienophile moiety are linked via a squarate group.
